(19) United States
(12) Patent Application Publication
(10) Pub. No.: US 2023/0270727 A1
(43) Pub. Date: Aug. 31, 2023

(54) USE OF VEGF INHIBITOR IN PREPARATION OF MEDICAMENT FOR TREATING HYPOXIA-RELATED DISEASES

(71) Applicants: Yihai CAO, Solna (SE); QILU HOSPITAL OF SHANDONG UNIVERSITY, Jinan, Shandong (CN)

(72) Inventors: Yuguo CHEN, Jinan, Shandong (CN); Yihai CAO, Solna (SE); Jiaojiao PANG, Jinan, Shandong (CN); Feng XU, Jinan, Shandong (CN); Ming LV, Jinan, Shandong (CN); Bailu WANG, Jinan, Shandong (CN); Yu LI, Jinan, Shandong (CN); Yuan BIAN, Jinan, Shandong (CN); Xiang JI, Jinan, Shandong (CN); Yuan ZHANG, Jinan, Shandong (CN); Wenhai SUI, Jinan, Shandong (CN)

(21) Appl. No.: 17/996,994

(22) PCT Filed: Apr. 23, 2021
(86) PCT No.: PCT/CN2021/089337

§ 371 Date: May 22, 2023

(30) Foreign Application Priority Data
Apr. 24, 2020 (CN) 202010335307.2

(51) Int. Cl. A61K 31/436; A61K 31/4745; A61K 31/404; A61K 31/4402; A61P 31/14; A61K 31/517; C07K 16/22; A61K 31/506

(52) U.S. Cl. CPC A61K 31/436; A61K 31/4745; A61K 31/404; A61K 31/4402; A61P 31/14; A61K 31/517; C07K 16/22; A61K 31/506

(57) ABSTRACT

A VEGF inhibitor is used in preparation of a medicament for treating hypoxia-related diseases. The VEGF inhibitor can significantly inhibit VEGF stress expression caused by hypoxia by acting on a binding pathway of VEGF and a VEGF receptor, is used for treating hypoxia and other related diseases, can significantly improve the oxygenation index of a patient, and can alleviate the hypoxic state of lung and other organ tissues, having good therapeutic effects.

### TECHNICAL FIELD

The present disclosure relates to the field of medicines, particularly to use of a VEGF (vascular endothelial growth factor) inhibitor, and more particularly to use of the VEGF inhibitor in preparing a medicament for treating hypoxia-related diseases.

### BACKGROUND

Hypoxia is a pathological condition in which tissues and cells lack adequate supply of oxygen. Hypoxia triggers a variety of physiological responses in humans and other mammals. For example, normal biological processes in cells are often impaired by hypoxia, and hypoxia also leads to upregulation of genes associated with many physiological processes such as angiogenesis and sugar metabolism. Hypoxia can occur at the level of the organism, for example, during periods of dyspnea or interrupted ventilation, or low availability of oxygen.

Dyspnea (respiratory distress) is a common factor causing hypoxia in an individual, and any factor causing diffuse damage to the lungs, such as trauma, septicemia, or viral or bacterial pneumonia, can cause dyspnea, which further causes hypoxia or hypoxemia in tissue cells and various hypoxic complications, such as damage to the heart, liver and kidneys.

It has been demonstrated in the prior art that under the action of many pathogenic factors, alveolar mononuclear macrophages and neutrophils produce complement C5a, tumor necrosis factor-α (TNF-α) and interleukin-1β (IL-1β) to initiate inflammatory cascade reactions, thus stimulating various cytokines in the lung, such as vascular endothelial growth factor (VEGF) and hypoxia-inducible factor-1 (HIF-1α). The release of a large number of inflammatory mediators and cytokines leads to dyspnea (Sun Zhongji et al., Cytokines and Inflammatory Mediators in Onset of Acute Respiratory Distress Syndrome, Chinese Critical Care Medicine March 2003, 15(3):186-189), in which VEGF is involved in various pathological processes, such as the induction of pulmonary vascular dysfunction, pulmonary inflammatory response, pulmonary edema, hemorrhage, sepsis, etc. Hypoxia caused by dyspnea is one of the major symptoms of the epidemic outbreak of novel coronavirus disease COVID-19 (or corona virus disease 2019) caused by the novel coronavirus SARS-CoV-2. The viral infection in lung epithelial cells generally causes non-cardiogenic pulmonary edema and hyaline membrane. The manifestations comprise diffuse alveolar damage in proliferative stage or organizing stage with severe dyspnea, which is the leading factor of prolonged disease course and poor prognosis. Severe respiratory failure and refractory hypoxemia usually result in severe hypoxia, and multiple organ failure is the main cause of death. It was found that there are significant differences in the expression levels of many cytokines in blood of patients infected with the novel coronavirus compared to healthy individuals (Huang C, Wang Y, Li X, et al. Clinical features of patients infected with 2019 novel coronavirus in Wuhan, China. Lancet 2020, published online January 24.). However, treating or alleviating hypoxia through related regulation of cytokine levels has yet been reported.

### SUMMARY

The present disclosure provides use of a VEGF (vascular endothelial growth factor) inhibitor in treating a disease or condition, wherein the disease or condition is selected from a hypoxia-related disease.

According to the present disclosure, the hypoxia-related disease is not specifically defined. It comprises symptoms that result in hypoxia or insufficient oxygen intake in a subject's body, or a lesion or injury caused by insufficient oxygen supply to cells, tissues or organs of the subject. According to the present disclosure, the hypoxia-related disease is at least one selected from respiratory distress syndrome, pneumonia, pulmonary edema, acute lung injury, ventilator-induced lung injury, smoking-induced lung injury, lung cancer, pathological apnea, ischemic heart disease, acute myocardial infarction (AMI), ischemic encephalopathy, ischemic stroke, ocular ischemic disease, ischemic optic neuropathy, inflammation, septicemia, renal failure, tissue fibrosis, bronchial dysplasia, fetal distress, postsurgical hypoxia, anemia, hypovolemia, rheumatoid arthritis, poisoning (e.g., carbon monoxide poisoning, heavy metal poisoning), ischemia reperfusion injury (e.g., limb, bowel and kidney ischemia), asphyxia and vascular embolism. For example, the hypoxia-related disease is a pulmonary disease caused by hypoxia, including but not limited to, respiratory distress syndrome, pneumonia, pulmonary edema and acute lung injury. For another example, the hypoxia-related disease is respiratory distress syndrome or a complication thereof caused by respiratory tract infection, acute lung injury, trauma or poisoning, and the complication comprises at least one selected from pulmonary edema, inflammatory response or inflammatory factor storm, sepsis and organ failure.

According to the present disclosure, the respiratory tract infection comprises viral pneumonia, bacterial pneumonia and fungal infection. In one embodiment, the viral pneumonia is severe or critical pneumonia caused by infection with any one or more of coronavirus SARS-CoV-2, SARS-Cov or MERS-Cov.

The present disclosure further provides use of a VEGF inhibitor in treating pulmonary edema.

The present disclosure further provides use of a VEGF inhibitor in alleviating an inflammatory response or an inflammatory factor storm.

The present disclosure further provides use of a VEGF inhibitor in treating sepsis.

The present disclosure further provides use of a VEGF inhibitor in treating coronavirus disease COVID-19 or a condition arising therefrom. In one embodiment, the VEGF inhibitor is used for treating pneumonia or respiratory distress caused by COVID-19. In another embodiment, the VEGF inhibitor is used for treating pulmonary edema caused by COVID-19 infection. In another embodiment, the VEGF inhibitor is used for treating an inflammatory response or an inflammatory factor storm caused by COVID-19. In another embodiment, the VEGF inhibitor is used for treating a pulmonary exudative lesion caused by COVID-19 and increasing the oxygenation index of a subject. The present disclosure further provides use of a pharmaceutical composition comprising a VEGF inhibitor in treating the disease or condition described above. In one embodiment, the pharmaceutical composition further comprises at least one therapeutic agent that is an additional therapeutic agent active against the diseases described above.

The present disclosure further provides a method for treating the disease described above, comprising administering to a patient with the disease or condition described above a VEGF inhibitor.

The present disclosure further provides a VEGF inhibitor or a pharmaceutical composition comprising the VEGF inhibitor for use in treating the disease or condition described above.

The present disclosure further provides use of a VEGF inhibitor in preparing a medicament for treating a hypoxia-related disease.

According to the present disclosure, the hypoxia-related disease is at least one selected from respiratory distress syndrome, pneumonia, pulmonary edema, acute lung injury, ventilator-induced lung injury, smoking-induced lung injury, lung cancer, pathological apnea, ischemic heart disease, acute myocardial infarction (AMI), ischemic encephalopathy, ischemic stroke, ocular ischemic disease, ischemic optic neuropathy, inflammation, septicemia, renal failure, tissue fibrosis, bronchial dysplasia, fetal distress, postsurgical hypoxia, anemia, hypovolemia, rheumatoid arthritis, poisoning (e.g., carbon monoxide poisoning, heavy metal poisoning), ischemia reperfusion injury (e.g., limb, bowel and kidney ischemia), asphyxia and vascular embolism. For example, the hypoxia-related disease is a pulmonary disease caused by hypoxia, including but not limited to, respiratory distress syndrome, pneumonia, pulmonary edema and acute lung injury. For another example, the hypoxia-related disease is respiratory distress syndrome or a complication thereof caused by respiratory tract infection, acute lung injury, trauma or poisoning, and the complication comprises at least one selected from pulmonary edema, inflammatory response or inflammatory factor storm, sepsis and organ failure.

According to the present disclosure, the respiratory tract infection comprises viral pneumonia, bacterial pneumonia and fungal infection. In one embodiment, the viral pneumonia is severe or critical pneumonia caused by infection with any one or more of coronavirus SARS-CoV-2, SARS-Cov or MERS-Cov.

The present disclosure further provides use of a VEGF inhibitor in preparing a medicament for treating pulmonary edema.

The present disclosure further provides use of a VEGF inhibitor in preparing a medicament for alleviating an inflammatory response or an inflammatory factor storm.

The present disclosure further provides use of a VEGF inhibitor in preparing a medicament for treating sepsis.

The present disclosure further provides use of a VEGF inhibitor in preparing a medicament for treating coronavirus disease COVID-19 or a symptom arising therefrom. In one embodiment, the medicament is used for treating severe or critical pneumonia caused by COVID-19. In another embodiment, the medicament is used for treating respiratory distress caused by COVID-19 infection. In another embodiment, the medicament is used for treating pulmonary edema caused by COVID-19 infection. In another embodiment, the medicament is used for alleviating or reducing an inflammatory response or an inflammatory factor storm caused by a COVID-19 infection. In another embodiment, the medicament is used for reducing an exudative lesion caused by COVID-19 infection and increasing the oxygenation index of a subject.

“Hypoxia” used herein refers to a circumstance in which oxygen is deficient or in which the supply of oxygen is below a physiological level, including chronic hypoxia or acute hypoxia. In one embodiment, the hypoxia refers to an oxygenation index (PaO₂/FiO₂, in mmHg)≤300 mmHg and/or a fingertip pulse oxygen saturation in resting state without oxygen therapy ≤96%, for example ≤90%, for another example ≤85%, for still another example ≤80%.

In one embodiment, administration of the VEGF inhibitor results in an oxygenation index (PaO₂/FiO₂, in mmHg)≥300 mmHg, for example ≥330 mmHg, for another example ≥360 mmHg, in a subject. In one embodiment, administration of the VEGF inhibitor results in a fingertip pulse oxygen saturation in resting state without oxygen therapy ≥96%, for example ≥98%, for another example ≥99%, for still another example 100%, in a subject.

“Hypoxia-related disease” used herein comprises a condition in which a respiratory disorder in a patient leads to insufficient oxygen intake and reduced blood oxygen, or reduced blood flow to an organ results in an oxygen level in an organ, tissue or cell below a level or range required by normal physiological activity. Hypoxia may be a symptom, or plays a role in the cause, development, progression, amelioration or cure of a disease, disorder or condition. In one embodiment, hypoxia is caused by reduced oxygen intake in lungs, including dyspnea due to lung lesions or traumas, respiratory tract lesions or traumas or allergies, or asphyxia or respiratory disorders due to external factors, such as drowning, poisoning and the like. In one embodiment, hypoxia is caused by a pulmonary lesion, such as respiratory distress syndrome, chronic obstructive pulmonary disease, emphysema, bronchitis, pulmonary edema, pneumonia, acute lung injury, ventilator-induced lung injury, smoking-induced lung injury, lung cancer, pathological apnea and the like. In one embodiment, hypoxia is due to reduced blood flow to an organ, such as vascular embolism, vascular rupture, trauma, inflammation and the like. The hypoxia-related disease comprises, but is not limited to: respiratory distress syndrome, pneumonia, pulmonary edema, acute lung injury, ventilator-induced lung injury, smoking-induced lung injury, lung cancer, pathological apnea, ischemic heart disease, acute myocardial infarction (AMI), ischemic encephalopathy, ischemic stroke, ocular ischemic disease, ischemic optic neuropathy, inflammation, septicemia, renal failure, tissue fibrosis, bronchial dysplasia, fetal distress, postsurgical hypoxia, anemia, hypovolemia, rheumatoid arthritis, poisoning (e.g., carbon monoxide poisoning, heavy metal poisoning), ischemia reperfusion injury (e.g., limb, bowel and kidney ischemia), asphyxia, vascular embolism and the like.

Respiratory distress or dyspnea is manifested clinically as shortness of breath, difficulty in breath, suprasternal and substernal inspirational retractions, nasal flaring, expanded range of atelectasis and elevated severity of respiratory failure. Respiratory distress can be a dysfunction in the gas-liquid exchange in the vascular tissue in lungs due to various causes, resulting in severe hypoxemia and dyspnea. Respiratory distress syndrome described herein may be caused by any severe lung injury, including but not limited to dyspnea or respiratory failure due to infection by various pathogen infections, trauma or poisoning, such as pneumonia caused by various fungal, bacterial or viral infections, inhalation of toxic chemicals, septic shock, vomit inhalation and the like. In one embodiment, respiratory distress syndrome described herein is dyspnea caused by a lung infection including but not limited to, coronavirus SARS-Cov-2, SARS-Cov or SARS virus infection, MERS-Cov infection, various influenza virus (e.g., H1N1, H7N9 or other influenza viruses) infections, bacterial infection, fungal infection and the like. The coronavirus SARS-Cov-2 described herein is a member of Betacoronavirus, which comprises coronavirus having a gene sequence of Genebank ID of MN908947 or a high homology, for example, 98% homology or higher, to the sequence. COVID-19 and a condition caused by the same refer to a disease or lesion caused by infection of novel coronavirus SARS-Cov-2, including lung injury, respiratory distress syndrome, sepsis and the like of various severities. The COVID-19 can be diagnosed by RT-PCR etiological nucleic acid detection on a sample, serum specific antibody detection and/or lung CT imaging. The sample of etiological detection comprises, but is not limited to, an upper respiratory tract sample, a lower respiratory tract sample, a digestive tract sample, a body fluid sample and the like, for example, nasopharyngeal swab, sputum, stool, urine, blood, tear, sweat, saliva and the like.

The terms “subject” and “patient” used herein have the same meaning and refer to a human or other warm-blooded mammals. The human described herein as a “subject” comprises adults, infants and children, and the warm-blooded mammals comprise, but are not limited to, non-human primates such as chimpanzees, other apes or monkeys, other zoo animals, domestic animals or laboratory animals such as cats, pigs, dogs, cattle, sheep, mice, rats, and guinea pigs and the like. Preferably, the “subject” described herein is a human.

An important aspect of the present disclosure is that hypoxia is associated with upregulation of VEGF expression, which results in cellular hypoxia when a patient experiences dyspnea or ischemia, and a substantial increase in the transcription factor of hypoxia inducible factor HIF-1α. VEGF gene is the transcription target gene of HIF, and increased HIF-1α induces massive synthesis of VEGF, thus stimulating angiogenesis to compensate tissue hypoxia and further maintaining and improving tissue function. VEGF binds to VEGF receptors on endothelial cells, triggering the tyrosine kinase pathway to induce angiogenesis. According to the present disclosure, the VEGF inhibitor blocks VEGF stress response and expression caused by hypoxia, such that the VEGF inhibitor has a beneficial effect on the hypoxia-related disease and effectively treats various diseases caused by hypoxia.

Patients with severe or critical coronavirus infection may experience dyspnea, which results in severe hypoxia and thus up-regulation of VEGF, increased vascular permeability and pulmonary edema. Thus, treatment of respiratory distress syndrome comprises, but is not limited to, inhibiting the binding of VEGF to VEGF receptors or reducing VEGF expression, alleviating respiratory distress by inhibiting pulmonary edema, reducing interstitial fluid exudation, increasing oxygenation index, reducing inflammatory response or inflammatory factor storm or the like.

In the present disclosure, “severe pneumonia” means that the patient meets any of the following:
1. Respiratory distress with a respiratory rate (RR)≥30 times/min;
2. Fingertip pulse oxygen saturation in resting state without oxygen therapy ≤93%;
3. Partial arterial pressure of oxygen (PaO₂)/fraction of inspired oxygen (FiO₂)≤300 mmHg;
4. Those conforming to any one selected from the above are managed as severe cases; alternatively, the following cases that does not conform to the diagnosis criteria described above may also be managed as severe cases: radiologically confirmed significant progression in lungs >50% within 24-48 hours; aged >60 years with severe chronic diseases including hypertension, diabetes, coronary heart disease, malignancy, structural lung disease, pulmonary heart disease, immunosuppression or the like.

“Critical pneumonia” means that the patient meets any of the following:
1. Respiratory failure requiring mechanical ventilation;
2. Occurrence of shock;
3. Having other organ failure and requiring admission to ICU.

The VEGF inhibitor described herein is not specifically defined, and any substance that can inhibit VEGF expression or has an inhibitory effect on upstream or downstream of VEGF signaling pathway is effective for hypoxia-related diseases described herein, including but not limited to substances acting on mammalian target of rapamycin (mTOR) signaling pathway, substances acting on hypoxia inducible factor HIF-1α pathway, substances directly acting on vascular endothelial growth factor VEGF pathway, or substances acting on other cellular biological processes related to VEGF signaling pathway. The VEGF inhibitor may be a macromolecular drug, such as a monoclonal antibody or a polypeptide, or a gene therapy drug, such as a cloning vector or a micromolecular compound.

In one embodiment, the VEGF inhibitor is a substance targeting the interaction between VEGF and VEGFr (vascular endothelial growth factor receptor). The VEGF inhibitor described herein refers to a compound that is capable of inhibiting one or more biological activities of VEGF, such as the mitogenic or angiogenic activity. The VEGF inhibitor works by interfering with the binding of VEGF to cellular receptors, by blocking signaling upon VEGF receptor activation, by disabling or killing VEGF-activated cells, or by interfering with the activation process of vascular endothelial cells upon VEGF binding to cellular receptors. In one embodiment, the VEGF inhibitor described herein may be an anti-VEGF drug or an anti-VEGF receptor drug. In one embodiment, the VEGF inhibitor is an anti-VEGF antibody (e.g., bevacizumab), an antibody derivative (e.g., ranibizumab Lucentis) or an anti-VEGF peptide; in one embodiment, the VEGF inhibitor may be a gene therapy drug, for example, a microbial cloning vector expressing a VEGF antibody or a gene therapy drug inhibiting VEGF expression; in one embodiment, the VEGF inhibitor is a micromolecular VEGF receptor inhibitor, for example, lapatinib, sunitinib, sorafenib, axitinib, pazopanib and the like.

In one embodiment, the VEGF inhibitor is an mTOR inhibitor, and can act on an mTOR signaling pathway and affect the expression of downstream cytokine HIF-1α or VEGF, thereby realizing the regulation of VEGF. The mTOR inhibitor may be selected from a variety of macromolecular drugs, gene therapy drugs or micromolecular compounds known in the art to act on the mTOR signaling pathway; for example, the mTOR inhibitor is at least one selected from rapamycin and everolimus.

In one embodiment, the VEGF inhibitor is an HIF-1α inhibitor. In the condition of hypoxia, HIF-1α degradation is blocked and, when combined with HIF-1β, forms HIF-1 molecule. Increased expression of HIF-1α upregulates VEGF expression and thus significantly accelerates the growth of blood vessel, and thus the HIF-1α inhibitor can play a role in inhibiting the expression of VEGF. The HIF-1α inhibitor comprises, but is not limited to, inhibiting the expression of HIF-1α, accelerating HIF-1α degradation, affecting HIF-1α nuclear aggregation, blocking HIF-1α binding to HIF-1β, and the like; for example, the HIF-1α inhibitor comprises, but is not limited to, temsirolimus, topotecan, camptothecin and the like.

In one embodiment, the VEGF inhibitor described herein is preferably selected from bevacizumab. Bevacizumab described herein is disclosed in U.S. Pat. No. 6,884,879, which is incorporated herein by reference in its entirety for bevacizumab. Bevacizumab described herein comprises, but is not limited to, commercial or non-commercial bevacizumab formulations known in the art (e.g., Avastin), bevacizumab biosimilars with bioequivalence and consistency (e.g., Ankeda), or derivatives of bevacizumab.

A therapeutically effective amount of the VEGF inhibitor may be administered to a subject to achieve a therapeutic effect for a hypoxia-related disease. The “therapeutically effective amount” may be determined according to methods known to eligible physicians in the art. Determination of the therapeutically effective amount is within the capability of clinicians or researchers in the art. For example, the dose of bevacizumab can be determined according to the dose described in U.S. Pat. No. 6,884,879, in combination with the dose of commercial bevacizumab, and according to the specific situation of the subject. In one embodiment, bevacizumab is administered to an adult at a dose of 1-100 mg/kg per day, e.g., 10-50 mg/kg per day and 12-15 mg/kg per day, and may be administered in a single dose or multiple doses depending on the individual factors of the patient and the severity of the symptoms. Individual factors of a patient generally comprise age, body weight, general health condition and other factors that may affect the efficacy, such as drug allergy history.

The VEGF inhibitor described herein can be administered by any route known in the art, including but not limited to, intravenous injection, intramuscular injection, subcutaneous injection, oral administration, inhalation and the like. The route of administration can be determined by those skilled in the art depending on the situation of the patient. Accordingly, the VEGF inhibitor described herein or the pharmaceutical composition comprising the same may be formulated into various suitable dosage forms including tablets, capsules, pills, powders and the like, according to the route of administration. In one embodiment, the medicament is administered by intravenous injection.

The VEGF inhibitor may be co-administered with other modalities or pharmaceutics that alleviate hypoxia or hypoxia-related disease. For example, modalities such as mechanical ventilation can be given while a therapeutically effective amount of the VEGF inhibitor is administered to a patient; or other drugs effective against the corresponding disease while the VEGF inhibitor is administered to the patient concurrently, e.g., at least one selected from an antifungal agent, an antibacterial agent, an antiviral agent, an antithrombotic agent, an immunomodulatory agent, an eye drop, a urologic agent, a hormonal agent, an anti-infective agent, an anti-inflammatory agent and the like is administered in combination with the VEGF inhibitor. In one embodiment, two or more active pharmaceutical ingredients that are administered in combination or co-administered are contained in one pharmaceutical composition. In another embodiment, a single pharmaceutical composition is not necessary, and separate pharmaceutical compositions may be comprised; the same dosage form, the same route of administration or the same time for administering the VEGF inhibitor described herein and other combined drugs is not necessary neither. However, for convenience of administration, two or more active pharmaceutical ingredients to be administered in combination may be formulated in the same dosage form, and may be administered at substantially the same administration time.

“Treatment” used herein, when relating to a disease or condition, refers to preventing or stopping the worsening of the disease or condition by medical action conducted on the subject, at least maintaining the situation or alleviating, more preferably completely curing and resolving the disease or condition. Specifically, the “treatment” used herein comprises alleviating or eliminating the associated conditions caused by hypoxia in a patient by administration of a drug alone or in combination with other therapeutic measures. In one embodiment, the term “treating” or “treatment” refers to alleviating or eliminating respiratory distress symptoms and stabilizing respiratory indexes, including increasing the patient's oxygenation index and blood oxygen saturation and improving tissue oxygenation status. Also comprised are reducing pulmonary exudative lesions, promoting significant absorption of pulmonary lesions and reducing the total volume of pulmonary lesions.

### Advantageous Effects

The VEGF inhibitor acts on a binding pathway of VEGF and VEGF receptors, can significantly inhibit VEGF stress expression caused by hypoxia, and thus can be used for treating hypoxia and other related diseases. It can significantly improve the oxygenation index in a patient, alleviate the hypoxia in lung and other organ tissues, and improve the respiratory state and ischemic symptoms. Particularly, bevacizumab for conditions caused by COVID-19 can improve oxygenation index in a patient, significantly reduce the volume of pulmonary lesions, promote the absorption of pulmonary lesions, improve the immunity of the patient, inhibit inflammatory factor storm and promote tissue recovery, thus having good efficacy.

### BRIEF DESCRIPTION OF THE DRAWINGS

FIG. 1 illustrates a curve and a histogram showing oxygenation index changes in subjects in the treatment group before bevacizumab treatment, 1 day after bevacizumab treatment and 7 days after bevacizumab treatment;

FIG. 2 illustrates the lung CT imaging performance comparison of different subjects before and after treatment;

FIG. 3 illustrates a curve and a histogram showing lymphocyte count changes in subjects in the treatment group before bevacizumab treatment and 3 days after the treatment;

FIG. 4 illustrates curves showing hs-CRP and CRP changes in subjects in the treatment group before bevacizumab treatment and 3 days after the treatment; and

FIG. 5 illustrates a curve and a histogram showing lactate dehydrogenase changes in subjects in the treatment group before bevacizumab treatment and 3 days after the treatment.

### DETAILED DESCRIPTION

The present disclosure will be further illustrated with reference to the following examples, but the present disclosure is not limited thereto. The methods are conventional methods unless otherwise stated. The materials are available from published sources unless otherwise indicated.

Bevacizumab used in the examples of the present disclosure is an approved product Avastin.

### Protocol:

The study was conducted by Qilu Hospital of Shandong University after repeated verifications, ethical review and regulatory registration (NCT04275414).

Subject (P): Patients with severe or critical COVID-19 and radiologically confirmed exudative lesions; Intervention (I): bevacizumab 500 mg in 0.9% sodium chloride solution 100 mL, administered through a single intravenous drip within not less than 90 min combined with conventional treatment; Comparison (C): comparison between before and after bevacizumab treatment, and comparison with reference; Primary endpoint (O): oxygenation index, quantitative pulmonary lesion value (calculated by imaging software).

Subjects with possible COVID-19 should be subjected to nasopharyngeal swab RT-PCR COVID-19 viral nucleic acid test, combined with specific IgM antibody and IgG antibody serum test and pulmonary CT imaging for diagnosis. Patients were classified into severe and critical cases according to diagnosis criteria for severe and critical types in the “Diagnosis and Treatment Protocol for COVID-19” (trial version 5, revised version) issued by the National Health Committee, PRC:

### (I) Severe Case

The subject should meet any of the following:
1. Respiratory distress with a respiratory rate (RR)≥30 times/min;
2. Fingertip pulse oxygen saturation in resting state without oxygen therapy ≤93%;
3. Partial arterial pressure of oxygen (PaO₂)/fraction of inspired oxygen (FiO₂)≤300 mmHg;
4. Those conforming to any one of the above are managed as severe cases; alternatively, the following cases that does not conform to the diagnosis criteria described above may also be managed as severe cases: radiologically confirmed significant progression in lungs >50% within 24-48 hours; aged >60 years with severe chronic diseases including hypertension, diabetes, coronary heart disease, malignancy, structural lung disease, pulmonary heart disease, immunosuppression or the like.

### (II) Critical Case

The subject should meet any of the following:
1. Respiratory failure requiring mechanical ventilation;
2. Occurrence of shock;
3. Having other organ failure and requiring admission to ICU.

The total number of subjects in the treatment group was 11. The demographics and baseline characteristics of the patients in the treatment and control groups are shown in Table 1.

TABLE 1
Baseline characteristics of patients
in the treatment and control groups

| | Treatment | | Control | | |
|---|---|---|---|---|---|
| | group | | group | | |
| | (n = 11) | | (n =22) | | P |
| Age (years) | 60.1 ± 9.3 | | 61.9 ± 9.9 | | 0.615 |
| Highest body temperature (° C.) | 38.9 ± 0.8 | | 38.5 ± 0.7 | | 0.182 |
| Days from onset to admission | 10.5 ± 4.1 | | 11.8 ± 5.2 | | 0.467 |
| Gender | | | | | |
| Female | 3 | (27.3%) | 6 | (27.3%) | 1.000 |
| Male | 8 | (72.7%) | 16 | (72.7%) | |
| Medical History | | | | | |
| Heart disease | 0 | (0.0%) | 3 | (13.6%) | 0.534 |
| Hypertension | 2 | (18.2%) | 9 | (40.9%) | 0.258 |
| Diabetes | 0 | (0.0%) | 4 | (18.2%) | 0.276 |
| Chronic obstructive | 0 | (0.0%) | 0 | (0.0%) | 1.000 |
| pulmonary disease | | | | | |
| Symptoms | | | | | |
| Fever | 11 | (100.0%) | 19 | (86.4%) | 0.534 |
| Asthenia | 8 | (72.7%) | 11 | (50.0%) | 0.278 |
| Dry cough | 7 | (63.6%) | 12 | (54.5%) | 0.719 |
| Polypnea | 6 | (54.5%) | 5 | (22.7%) | 0.117 |
| Productive cough | 4 | (36.4%) | 5 | (22.7%) | 0.438 |
| Tightness in chest | 4 | (36.4%) | 6 | (27.3%) | 0.437 |
| Chills | 3 | (27.3%) | 4 | (18.2%) | 0.661 |
| Head pain | 3 | (27.3%) | 1 | (4.5%) | 0.097 |

### (1) Oxygenation Index

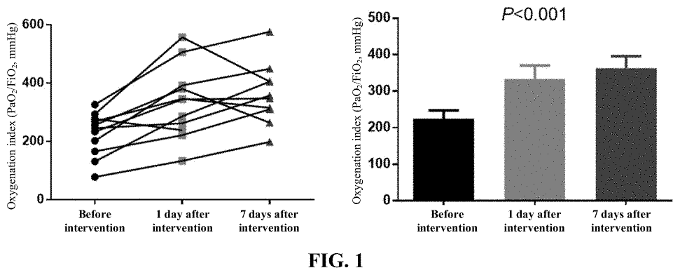

The oxygenation status of the tissues in the subjects was significantly improved on Days 1 and 7 after bevacizumab treatment (results are shown in Table 2 and FIG. 1), and the oxygenation index (PaO₂/FiO₂) was significantly increased (P<0.001) as compared to that before treatment.

TABLE 2
Changes in respiratory indexes in treatment
group before and after bevacizumab treatment

| | Before | 1 day after | 7 days after | |
|---|---|---|---|---|
| | intervention | intervention | intervention | |
| | (n = 11) | (n = 11) | (n = 10) | P |
| Oxygenation index | 225.0 ± | 333.2 ± | 362.6 ± | <0.001 |
| (PaO₂/FiO₂, mmHg) | 74.3 | 124.5 | 104.7 | |
| Oxygen saturation | 96.2 ± | 96.8 ± | 96.9 ± | 0.505 |
| (SpO₂, %) | 5.3 | 3.2 | 2.4 | |
| Partial pressure | 125.4 ± | 108.1 ± | 103.1 ± | 0.467 |
| of oxygen | 58.5 | 39.3 | 36.6 | |
| (PaO₂, mmHg) | | | | |

### (2) Lung CT Quantitative Analysis and Radiological Representation

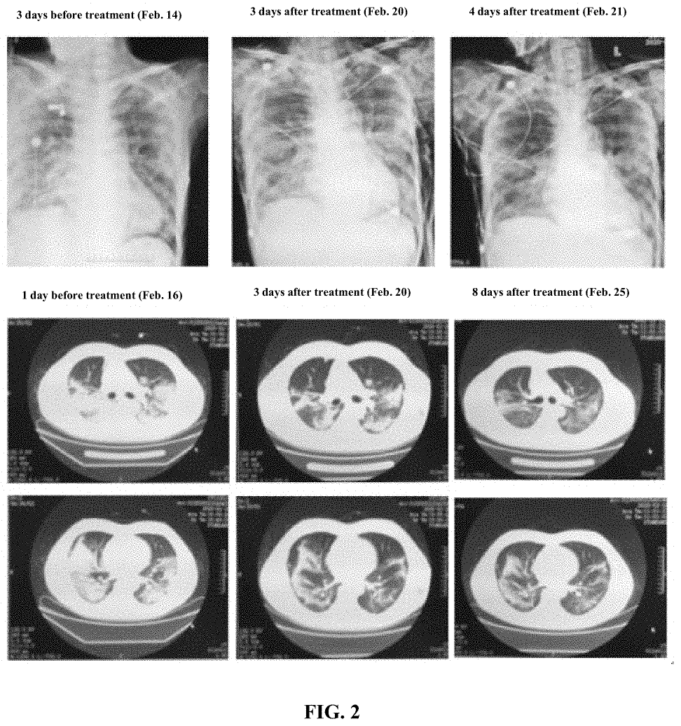

Taking pre-dose CT images as the initial point, the CT images 7 days after the bevacizumab treatment were compared and analyzed. Quantitative lung CT analysis showed that bevacizumab treatment significantly promoted absorption of lung lesions within 7 days. After bevacizumab treatment, the number of patchy opacifications decreased significantly (P=0.024) and some shifted to milder ground glass opacifications after absorption (P=0.007); the total lesion volume decreased significantly (P=0.028), and proportion of lesions in the left (right) lung decreased (Table 3). The changes reflected by the above quantitative analysis can be visually observed through the lung images, suggesting significant efficacy (FIG. 2).

TABLE 3
Lung CT quantitative analysis in treatment group before and after bevacizumab treatment

| | Initial point | | 7 d | | |
|---|---|---|---|---|---|
| | (n = 10) | | (n = 10) | | P |
| Number of patchy opacifications | 2.5 | (0, 8) | 0 | (0, 6) | 0.024 |
| Number of ground glass opacifications | 3 | (0, 9) | 7 | (2, 18) | 0.007 |
| Total volume of lesion (cm³) | 785.9 | (362.3, 1554.4) | 568.9 | (374.4, 1372.0) | 0.028 |
| Proportion of lesions in right lung (%) | 27.2 | (7.9, 65.2) | 14.0 | (6.3, 76.1) | 0.074 |
| Proportion of lesions in left lung (%) | 31.4 | (7.3, 60.0) | 14.6 | (6.2, 41.7) | 0.093 |

### (3) Immunity

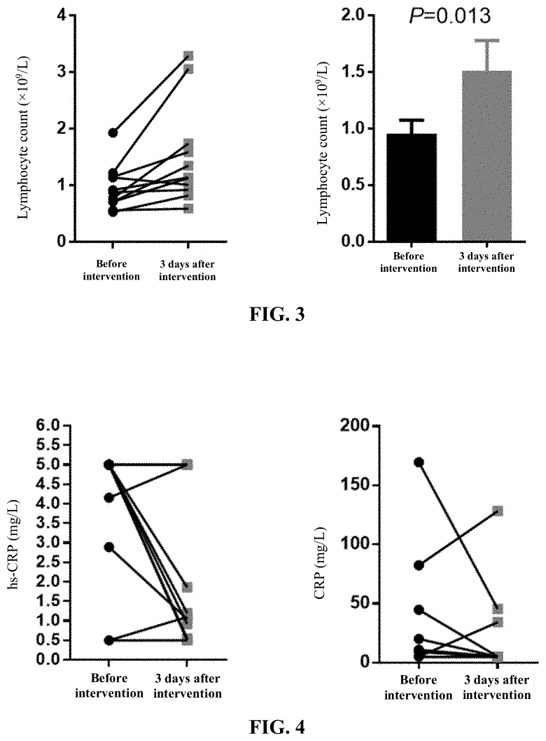

Lymphocyte in the subjects were significantly increased 3 days after bevacizumab treatment (P=0.013), suggesting that the immune status was improved (results are shown in Table 4 and FIG. 3).

TABLE 4
Comparison of complete blood counts in treatment
group before and after bevacizumab treatment

| | Before | 3 days after | |
|---|---|---|---|
| | intervention | intervention | |
| | (n = 11) | (n = 11) | P |
| White blood cell count (×10⁹/L) | 6.6 (3.6, 19.2) | 7.3 (3.1, 15.8) | 0.722 |
| Neutrophil count (×10⁹/L) | 4.3 (1.5, 18.2) | 4.3 (1.4, 13.2) | 0.722 |
| Lymphocyte count (×10⁹/L) | 1.0 ± 0.4 | 1.5 ± 0.9 | 0.013 |
| Platelet count (×10⁹/L) | 243.3 ± 92.1 | 231.3 ± 100.1 | 0.589 |
| Red blood cell count (×10¹²/L) | 4.1 ± 0.7 | 4.0 ± 0.7 | 0.120 |
| Hemoglobin (g/L) | 122.0 ± 17.9 | 117.3 ± 20.4 | 0.055 |

### (4) Inflammatory Factors

hs-CRP level decreased significantly as compared to the baseline 3 days after bevacizumab treatment (P=0.036); CRP also exhibited a descending trend (results are shown in Table 5 and FIG. 4).

TABLE 5
Changes in hs-CRP and CRP in treatment group
before and after bevacizumab treatment

| | Before | 3 days after | |
|---|---|---|---|
| | intervention | intervention | |
| | (n = 11) | (n = 11) | P |
| hs-CRP (mg/L) | 5.0 (0.5, 5.0) | 1.1 (0.5, 5.0) | 0.036 |
| CRP (mg/L) | 9.2 (5.0, 169.5) | 5.0 (5.0, 128.2) | 0.401 |

### (5) Lactate Dehydrogenase (LDH)

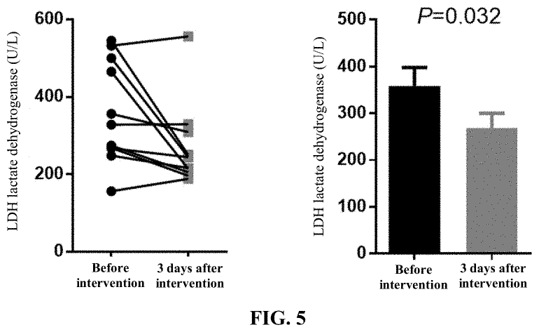

LDH decreased significantly (P=0.032) 3 days after bevacizumab treatment as compared to the baseline, suggesting a recovery trend of tissue injury (results are shown in Table 6 and FIG. 5).

TABLE 6
Changes in LDH in treatment group before
and after bevacizumab treatment

| | Before | 3 days after | |
|---|---|---|---|
| | intervention | intervention | |
| | (n = 11) | (n = 11) | P |
| LDH lactate | 358.1 ± 131.9 | 268.3 ± 105.4 | 0.032 |
| dehydrogenase (U/L) | | | |

The results showed that: for patients with severe and critical COVID-19, after bevacizumab treatment, the oxygenation index was significantly improved; lung CT quantitative analysis showed significantly reduced volume of lesions, significantly reduced proportion of lesions and shift from patchy opacifications to milder ground glass opacifications; lymphocyte count (L) was increased, indicating improvement of immunity; a plurality of important indexes including high-sensitivity C-reactive protein (hs-CRP) and lactate dehydrogenase (LDH) were significantly improved; adverse events such as allergy, hemoptysis, gastrointestinal hemorrhage, neutropenia and the like were not found in any patients during the treatment.

### (6) Matching Analysis of Treatment and Control Groups

The treatment group and the control group showed no statistical significance (P>0.05) in the aspects of age, highest body temperature, days from onset to admission, gender, heart disease history, hypertension history, diabetes history, chronic obstructive pulmonary disease history and symptoms such as fever, asthenia, dry cough and the like. The two groups were comparable in the aspect of baseline data (shown in Table 1). In the important indexes, the proportion of significantly improved oxygenation indexes (increased by 100 mmHg) in the treatment group receiving bevacizumab and the improvements in hs-CRP and lymphocyte count were higher than those in the control group, and other indexes demonstrated no significant difference (Table 7).

TABLE 7
Comparison of improvement in important
indexes of treatment and control groups

| | Treatment | Control | |
|---|---|---|---|
| | group | group | |
| | (n = 11) | (n = 22) | P |
| Ratio of significant improvements in | 55% | 25% | 0.183 |
| oxygenation index | | | |
| Median difference in lymphocyte count | 0.42 | 0.14 | 0.132 |
| Median difference in hs-CRP | −1.8 | 0.0 | 0.180 |
| Median difference in CRP | −4.0 | 0.0 | 0.432 |
| Median difference in LDH | −47.0 | −42.0 | 0.890 |

The results showed that compared with the control group, bevacizumab significantly improved the oxygenation index and alleviated respiratory failure symptoms in patients with severe and critical COVID-19 in the treatment group.

The exemplary embodiments of the present disclosure have been described above. However, the scope of the present disclosure is not limited to the above embodiments. Any modifications, equivalents, improvements and the like made by those skilled in the art without departing from the spirit and principle of the present disclosure shall fall within the protection scope of the present disclosure.

1. A method for treating a hypoxia-related disease, comprising administering a VEGF (vascular endothelial growth factor) inhibitor to a subject in need thereof.

2. The method according to claim 1, wherein the hypoxia-related disease comprises a pulmonary injury or symptom causing hypoxia or insufficient oxygen intake in lungs of a subject's body, or a lesion or injury due to insufficient oxygen supply to cells, tissues or organs of the subject; for example, the hypoxia-related disease comprises a pulmonary disease caused by hypoxia.

3. The method according to claim 1, wherein the hypoxia-related disease is at least one selected from respiratory distress syndrome, pneumonia, pulmonary edema, acute lung injury, ventilator-induced lung injury, smoking-induced lung injury, lung cancer, pathological apnea and asphyxia.

4. The method according to claim 1, wherein the hypoxia-related disease is at least one selected from ischemic heart disease, acute myocardial infarction (AMI), ischemic encephalopathy, ischemic stroke, ocular ischemic disease, ischemic optic neuropathy, inflammation, septicemia, renal failure, tissue fibrosis, bronchial dysplasia, fetal distress, postsurgical hypoxia, anemia, hypovolemia, rheumatoid arthritis, poisoning (e.g., carbon monoxide poisoning, heavy metal poisoning), ischemia reperfusion injury (e.g., limb, bowel and kidney ischemia) and vascular embolism.

5. The method according to claim 1, wherein the hypoxia-related disease is respiratory distress syndrome or a complication thereof caused by respiratory tract infection, acute lung injury, trauma or poisoning.

6. The method according to claim 5, wherein the complication comprises at least one selected from pulmonary edema, inflammatory response or inflammatory factor storm, sepsis and organ failure.

7. The method according to claim 5, wherein the respiratory tract infection comprises at least one selected from viral pneumonia, bacterial pneumonia and pulmonary fungal infection.

8. The method according to claim 7, wherein the viral pneumonia is severe or critical pneumonia caused by infection with any one or more of coronavirus SARS-CoV-2, SARS-Cov or MERS-Cov.

9. The method according to claim 1, wherein the VEGF inhibitor is a substance capable of inhibiting VEGF expression or a pathway thereof; preferably, the VEGF inhibitor is a substance targeting the interaction between VEGF and VEGFr (vascular endothelial growth factor receptor);
preferably, the VEGF inhibitor is an mTOR inhibitor, such as a macromolecular drug, a gene therapy drug or a micromolecular compound of the mTOR signaling pathway; for example, the mTOR inhibitor is selected from at least one selected from rapamycin and everolimus;
preferably, the VEGF inhibitor is an HIF-1α inhibitor; for example, the HIF-1α inhibitor is at least one selected from temsirolimus, topotecan and camptothecin.

10. The method according to claim 1, wherein the VEGF inhibitor is an anti-VEGF antibody, an antibody derivative or an anti-VEGF peptide; for example, the VEGF inhibitor is bevacizumab or ranibizumab.

11. The method according to claim 1, wherein the VEGF inhibitor is a gene-based drug; for example, the VEGF inhibitor is a microbial cloning vector expressing a VEGF antibody or a gene-based drug inhibiting VEGF expression.

12. The method according to claim 1, wherein the VEGF inhibitor is a micromolecular VEGF receptor inhibitor compound; for example, the VEGF inhibitor is any one selected from lapatinib, sunitinib, sorafenib, axitinib and pazopanib.

13. The method according to claim 1, wherein the hypoxia comprises chronic hypoxia or acute hypoxia.

14. The method according to claim 1, wherein a subject with hypoxia-related disease has an oxygenation index (PaO₂/FiO₂, in mmHg)≤300 mmHg and/or a fingertip pulse oxygen saturation in resting state without oxygen therapy ≤96%, for example ≤90%, for another example ≤85%, for still another example ≤80%.

15. The method according to claim 1, wherein administration of the VEGF inhibitor results in an oxygenation index (PaO₂/FiO₂, in mmHg)≥300 mmHg, for example ≥330 mmHg, for another example ≥360 mmHg, in a subject.

16. The method according to claim 1, wherein administration of the VEGF inhibitor results in a fingertip pulse oxygen saturation in resting state without oxygen therapy ≥96%, for example ≥98%, for another example ≥99%, for still another example 100%, in a subject.

17-27. (canceled)

28. A pharmaceutical composition comprising the VEGF inhibitor of claim 1.

29. The pharmaceutical composition according to claim 28, wherein the VEGF inhibitor is bevacizumab.

30. The pharmaceutical composition according to claim 28, wherein the pharmaceutical composition further comprises at least one therapeutic agent selected from an antifungal agent, an antibacterial agent, an antiviral agent, an antithrombotic agent, an immunomodulatory agent, an eye drop, a urologic agent, a hormonal agent, an anti-infective agent and an anti-inflammatory agent.

31. The method according to claim 1, wherein the hypoxia-related disease is caused by COVID-19.
